(19) United States
(12) Patent Application Publication
(10) Pub. No.: US 2023/0338676 A1
(43) Pub. Date: Oct. 26, 2023

(54) INTRANASAL DEVICE FOR AUTOMATIC OPIOID OVERDOSE DETECTION AND NALOXONE DELIVERY

(71) Applicant: Asher Varon, Baltimore, MD (US)

(72) Inventor: Asher Varon, Baltimore, MD (US)

(21) Appl. No.: 18/139,156

(22) Filed: Apr. 25, 2023

Related U.S. Application Data
(60) Provisional application No. 63/334,444, filed on Apr. 25, 2022.

(51) Int. Cl. A61M 15/08; A61M 15/00

(52) U.S. Cl. CPC A61M 15/085; A61M 15/0086; A61M 15/0098; A61M 2202/0275; A61M 2205/3303

(57) ABSTRACT

A wearable-intranasal device for opioid overdose detection via overdose-associated increases of carbon dioxide in exhaled breath, and automatic delivery of naloxone in response. The device comprises a wearable nasal clip with drug reservoir(s) containing naloxone, and a CO₂-sensitive hydrogel occluding or encapsulating the naloxone. The hydrogel transitions from a solid state to a liquid or gas state when exposed to an increase in CO₂, thereby unblocking the drug reservoir(s) and releasing naloxone. The device provides a novel disposable, stimuli-responsive, naloxone delivery system, designed to trigger automatically in response to physical symptoms of an overdose without the need for bystander intervention.

### CROSS-REFERENCE TO RELATED APPLICATION(S)

The present application derives priority from U.S. Provisional Pat. Application 63/334,444 filed 25 Apr. 2023.

### BACKGROUND OF THE INVENTION

### 1. Field of the Invention

The present invention relates generally to automated sensing of symptoms initiating drug delivery, and more particularly, to an intranasal device that can automatically detect opioid overdose and deliver naloxone to reverse the effect, thereby creating a safety-net for those at risk of opioid relapse.

### 2. Description of the Background

Since 1999, the number of yearly deaths due to opioid overdose has increased by 300%, with an estimated annual economic burden of $78.5 billion in the United States and an average individual cost of $30,000. 1000 individuals in the United States alone are treated in emergency departments daily for opioid abuse, costing $6,000 per visit with 4% of patients dying each day. Over the course of the next ten years it is predicted opioid overdoses will kill over half a million Americans placing the estimated social cost at $221.6 billion to $549.8 billion.

For those suffering from opioid use disorder (OUD), treatment remains difficult, long, and potentially dangerous. It has been estimated that an average patient will relapse 5-6 times while in substance-abuse treatment and studies have shown that over 90% of users enrolling in treatment will relapse. At these moments of relapse, OUD individuals are most susceptible to overdose. Strang, J. et al., Loss of Tolerance and Overdose Mortality After Inpatient Opiate Detoxification: Follow Up Study, BMJ 326, 959-960 (2003). As a result, OUD treatments are a growing market with the potential to mitigate the increasing social and economic costs of the epidemic. OUD treatments span a range of different drugs and therapies, from traditional methadone treatments to harm reduction techniques, such as needle exchanges, but one of the most important treatments is the overdose reversal drug, naloxone.

To combat opioid overdoses, naloxone is an emergency antidote drug for opioid overdose that when used properly by a bystander, reverses the effects of opioids in seconds, restoring normal respiration to a patient. Naloxone has been employed by various organizations such as emergency personnel, OUD treatment facilities, and community centers in an attempt to curb fatal outcomes. Yet while the drug itself targets the biological mechanism of overdose, acting as an opioid receptor antagonist, it fails to address the epidemiological problem of identifying and delivering treatment in a timely manner, since it generally requires both a bystander’s recognition of overdose and ability to administer it. If left untreated, overdose conditions can cause irreversible neural damage only four (4) minutes after onset, shorter than the national average emergency medical service response time of seven (7) minutes. There are existing delivery mechanisms for naloxone. See, e.g., U.S. Pat. Nos. 11,191,934 and 11,278,709. However, a need exists for automatic detection of opioid overdose and subsequent automatic delivery of naloxone to treat both those not pursuing treatment and those most vulnerable to fatal relapse.

There are several ongoing attempts to develop active overdose detection devices, some involving implantable devices and some electronically activated wearables. For example, Northwestern University’s Rogers Research group is developing an implant to actively monitor blood pH for overdose detection and naloxone administration. See, Morris, A., Bioelectronic Implant Could Prevent Opioid Deaths, Northwestern Engineering https://www.mccormick.northwestern.edu/news/articles/2019/10/bioelectronic-implant-could-prevent- opioid-deaths-john-rogers.html (2019). These attempts to date monitor various overdose metrics. For example, some rely on external wearable ECG sensors and respiratory monitors. The external devices tend to perform naloxone delivery by an auto injector, similar to wearable diabetic management systems for intramuscular administration. The implantable devices such as Northwestern University’s tend to rely on micropumps. Other relevant companies in this space include the following: Biosensics™, Rescue Biomedical™, Resilient Lifesciences™, Naloxone Pocket Corps™ (nasal swab); Masimo™ Opioid Halo (detection only). The gold standard for overdose detection in the field still remains carbon dioxide. Taghizadieh A., Mohammadinasab R., Sharbaf J.G., Michaleas S.N., Vrachatis D., Karamanou M. Theriac in the Persian traditional medicine. Erciyes Med. J. 2020;42(2):235-238. doi: 10.14744/etd.2020.30049. CO₂ in the blood correlates reliably with ventilation, and so a measurement of CO₂ processed by the lungs can directly indicate respiratory distress. Comparison of end-tidal carbon dioxide and arterial blood bicarbonate levels in patients with metabolic acidosis has been considered for emergency medicine applications. Aminiahidashti et a., Applications of End-Tidal Carbon Dioxide (ETCO₂) Monitoring In Emergency Department; A Narrative Review, Journal of Cardiovascular and Thoracic Research 8, 98-101 (2016). Increasing CO₂ at exhalation due to respiratory distress acidifies aqueous solutions as illustrated in FIG. 1, which illustrates that normal exhaled breath produces a pH change of around 1 unit visible in a buffered aqueous solution.

To date, no intranasal delivery method has been paired with a complimentary CO₂ detection system. What is needed is a wearable, disposable, intranasal naloxone delivery device that uses an integrated hydrogel biosensor to automatically detect respiratory distress by nasal CO₂ levels in exhaled breath, and an integral delivery mechanism to release a proper dose of naloxone when needed. This will provide a convenient voluntary prophylactic device for those with opioid use disorder susceptible to relapse.

### SUMMARY OF THE INVENTION

It is, therefore, an object of the present invention to provide an intranasal device for automatic opioid overdose detection and naloxone delivery, designed to trigger automatically in response to physical symptoms of an overdose without the need for bystander intervention.

According to the present invention, the above-described and other objects are accomplished by providing an intranasal device with integrated biosensor and delivery mechanism for automatic opioid overdose detection and naloxone delivery. The wearable device automatically detects opioid overdose, via overdose-associated increases of carbon dioxide in exhaled breath, and independently delivers naloxone.

Two embodiments are disclosed: 1) a nasal clip with sieve-like naloxone-containing chambers, where a hydrogel embeds or encapsulates the naloxone and phase-changes in response to CO₂ after which it flows out of the chambers and on to the mucosa; 2) a nasal clip with two enclosed chambers loaded with naloxone and pressurized, each chamber having an outlet occluded by a hydrogel. Once the hydrogel changes shape or phase transitions then the drug becomes expelled due to pressure.

In the first embodiment, the device comprises a wearable nasal clip generally U-shaped with two prongs, and a distal sieve-like structure at each prong that contains the biosensor: a CO₂ sensitive hydrogel encapsulating naloxone. The wearable acts an applicator holding the hydrogel, which is then directly exposed to exhaled breath form the nasal passageways in contact with the inferior nasal mucosa. Each prong of the wearable contains one dose of naloxone, for a total of two doses for each wearable device, in order to introduce redundancy and to account for overdose scenarios where an additional dose of naloxone is required. Once an overdose occurs, the gel solubilizes flowing onto the mucosa, delivering naloxone in a similar manner to current naloxone applicators such as nasal swabs.

In a second embodiment, the device comprises a wearable nasal clip generally U-shaped with two prongs, and two distal reservoir chambers on each end with an aerosolizing nozzle. The chambers are loaded with naloxone (powder or liquid) drug and pressure-loaded. The nozzle is filled with the biosensor: a material that occludes the aerosolizing nozzle. The material is phase or shape transitioning hydrogel, sensitive to CO₂. The hydrogel biosensor is configured to transition from its equilibrium state to a liquid, shrunken, or swollen state when exposed to an increase in CO₂, thereby unblocking said drug reservoir.

In all embodiments the device provides a novel disposable, stimuli-responsive, naloxone delivery system, designed to trigger automatically in response to physical symptoms of an overdose without the need for bystander intervention.

### BRIEF DESCRIPTION OF THE DRAWINGS

Other objects, features, and advantages of the present invention will become more apparent from the following detailed description of the preferred embodiments and certain modifications thereof when taken together with the accompanying drawings in which.

FIG. 1 illustrates the progressive shift of pH from 7 to 6 from prolonged exhaled breath expressed as color shifts with methylene blue.

FIG. 2 is a perspective view of an embodiment of the device 2.

FIG. 3 is a front view of the device 2 of FIG. 2.

FIG. 4 is a side view of the device 2 of FIGS. 2-3.

FIG. 5 is a top view of the device 2 of FIGS. 2-4.

FIG. 6 illustrates an alternate embodiment of the device 20

FIG. 7 illustrates a sequential method of use of devices 2, 20 and mechanism of CO₂ activation.

### DETAILED DESCRIPTION OF THE PREFERRED EMBODIMENT

Reference will now be made in detail to preferred embodiments of the present invention, examples of which are illustrated in the accompanying drawings. Wherever possible, the same reference numbers will be used throughout the drawings to refer to the same or like parts.

The present invention generally comprises an intranasal device with integrated biosensor and delivery mechanism for automatic opioid overdose detection and naloxone delivery. In an embodiment, the device is a noninvasive, disposable, wearable device that integrates a CO₂ biosensor with a naloxone delivery mechanism to release a proper dose of naloxone when needed. The device is worn on the nose voluntarily as a prophylactic for people with opioid use disorder susceptible to relapse. The biosensor is sensitive to pH changes and produces a phase transition from a solid or semi-solid (e.g., gel) state to a non-solid (e.g., liquid) state when exposed to an increase in CO₂. In equilibrium gel state the biosensor embeds, contains and/or blocks release of naloxone, but induction of phase or shape transition in the gel releases a prescribed amount of naloxone or drug into the nasal passages.

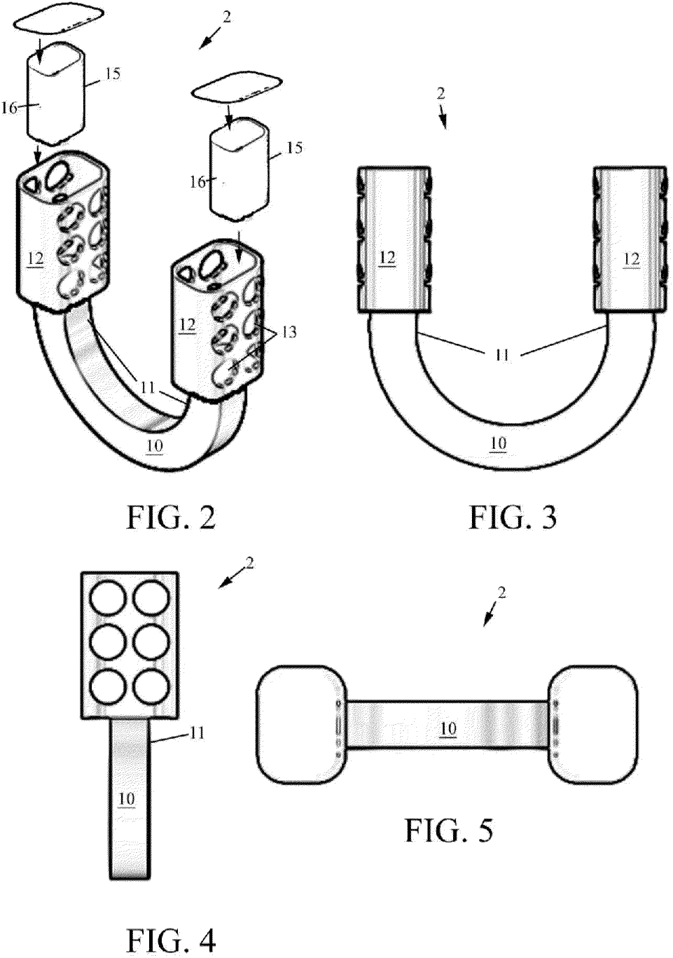

FIGS. 2-5 are a perspective view, front view, side view and top view, respectively, of an embodiment of the device 2. The device 2 comprises a wearable nasal clip generally U-shaped with a unitary two-pronged housing 10 with spaced prongs 11 that grip the septum, so that the device can be worn comfortably. Opposing drug reservoir chambers 12 are configured distally on each prong 11, each drug reservoir chamber 12 comprising a perforated sieve-like structure that contains a dose of naloxone 16 embedded, encapsulated or otherwise occluded by a phase-transition biosensor 15. The biosensor 15 may, for example, be a CO₂ sensitive hydrogel embedding, encapsulating and/or otherwise occluding the naloxone 16. The hydrogel biosensor 15 is configured to phase-transition when exposed to an increase in CO₂, thereby releasing or unblocking the naloxone 16. The unitary two-pronged housing 10 with spaced prongs 11 may be manufactured from thermoplastics and envisioned to produce a footprint of 10-30 mm (length) × 10-40 mm (height) × 2-10 mm (thick), and may be inserted by an individual directly prior to opioid use at the base of their septum and remains in place like a clip. This way, the wearable device 2 acts an applicator holding the hydrogel biosensor 15 in position for direct exposure to exhaled breath from the nasal passageways, breath that has been in contact with the inferior nasal mucosa. Once an overdose occurs, the hydrogel biosensor 15 solubilizes in response to CO₂/pH change, itself flowing onto the mucosa and delivering the naloxone 16 as effectively as conventional applicators such as nasal swabs. It is envisioned that the device 2 will be used as a convenient prophylactic device worn voluntarily by people with opioid use disorder most susceptible to relapse.

The two opposing drug reservoir chambers 12 may combine to deliver a single dose of naloxone, or alternatively each may contain a single dose of naloxone (for a total of two doses) in order to introduce redundancy and to account for overdose scenarios where an additional dose of naloxone is required.

During opioid overdose, opioids bind to receptors which activate inhibitory neurotransmitter signals, reducing respiratory function and eventually leading to cessation of respiration. With decreased respiration, oxygen and carbon dioxide (CO₂) exchange is reduced, polluting the bloodstream and capillaries with excess CO₂. This acidifies the cellular environment, normally held at a pH of 7.35-7.45. Buildup of CO₂ within the body, accompanied by hypoventilation in cases of reduced respiration, decreases homeostatic pH, causing respiratory acidosis. In an attempt to expel the excess amounts of CO₂ in the body during respiratory distress, the partial pressure of CO in exhaled breath increases from about 4.6 kPa to 6.0 kPa (>45 mm Hg, >6% overall concentration of CO₂ in exhaled breath) or higher. This increase in the partial pressure of CO₂ in exhaled breath correlates to a decreases in the pH of any aqueous solution the breath is exposed to. This is because dissolution of CO₂ into water produces carbonic acid (H₂CO₃).

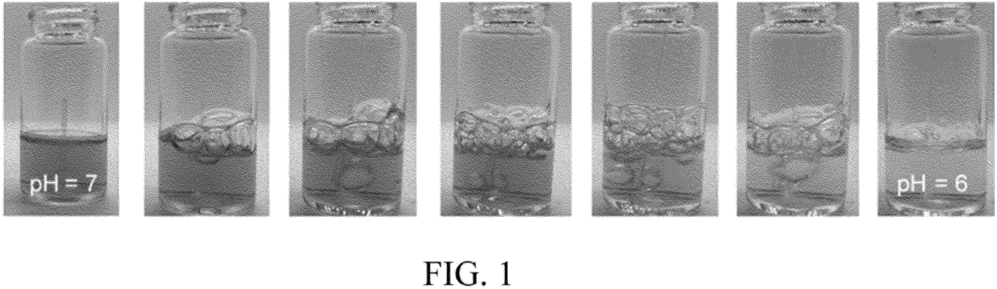

H₂CO₃ further dissociates into free floating hydrogens (H⁺) and bicarbonate (HCO₃⁻), which can decrease the pH of any exposed neutral solution. Thus, in the case of overdose-associated distress with excess CO₂, the pH level in a small volume decreases by at least 0.5 pH units, and typically around 1 unit (as seen in the buffered aqueous solution of FIG. 1). Consequently, the hydrogel biosensor 15 comprises any suitable material configured to transition from a solid or semi-solid (gel) state to a gas or liquid state when exposed to an increase in CO₂. In an embodiment sensor 15 comprises an aqueous hydrogel capable of absorbing CO₂ and configured to transition to liquid or gas state upon a measurable decrease in pH, preferably within a range of from 0.5 to 1 pH units.

Thus, for example, the hydrogel biosensor 15 may be an ABA Block Copolymer, or any other suitable substance capable of switching from insoluble to soluble in response to decreasing pH. Suitable ABA Block Copolymers include upper critical solution temperature (UCST) and lower critical solution temperature (LCST) ABA Block Copolymer Hydrogels.

For example, p(AAm-co-AN-co-DEAEMA)-b-PDMA-b-p(AAm-co-AN-co-DEAEMA), prepared by free radical precipitation polymerization of acrylamide (AAM), acrylonitrile (AN) and 2-(diethylamino)ethyl methacrylate (DEAEMA)hydrogels with responsive dissolution occurring at an upper critical solution temperature (UCST) or a lower critical solution temperature (LCST). The UCST represents the temperature at which a polymer is soluble at any mixing ratio of polymer to solvent and below which the polymer is not soluble at high concentration. UCSTs occur in polymers due to attractive ionic or hydrogen bonding interactions between polymer chains. At low temperatures, these attractive interactions prevent dissolution of the polymer by water. As temperature increases, the increase in thermal energy allows ionic and hydrogen bonds between chains to break, causing dissolution. In the case of p(AAm-co-AN) copolymers as above, hydrogen bonding between acrylamide monomers is responsible for the UCST. The device’s biosensor 15 maintains an initial UCST above 45° C., which causes the copolymer to remain insoluble before overdose. After acidification, the copolymer’s UCST decreases to below 2° C., rapidly dissolving the copolymer. The pH sensitivity of the UCST is a result of the weak polycation, 2-(diethylamino)ethyl methacrylate (DEAEMA). As pH decreases, the protonation state of DEAEMA changes, with the tertiary amine picking up a proton and becoming charged. This change in protonation state changes the enthalpy of solvation of the polymer, which causes a shift in the UCST. The shift is especially pronounced near the pKa of the polycation. DEAEMA was chosen as the weak polycation monomer because of its near neutral pKa, which lies around normal breath pH, relative biocompatibility, suitable for a biosensor. One skilled in the art will understand that other polymers composed of monomers with other neutral tertiary amine groups can function similarly, albeit at different pKa’s, the most common being methacrylate derivatives. Hydrogen bonding UCST polymers are particularly sensitive to pH due to their low, endothermic enthalpy of solvation. The small magnitude of this enthalpy means that changes to the protonation state of the weak polyelectrolyte have a large impact on the enthalpy of solvation and therefore the UCST. Substantial UCST shifts have been observed over pH changes as small as 0.05 pH units. In one existing literature example, a p(AAm-co-AN-co-4-vinylpyridine) copolymer was created and its cloud point was measured as a function of pH. The cloud point of one copolymer was found to shift by 57° C. due to a 0.25 change in pH, from 72° C. at pH 4.75 to 15° C. at pH 4.50. Further, the magnitude of this response was tunable. Decreasing the amount of the pH-responsive monomer, 4-vinylpyridine (4VP), in the copolymer from 10% to 5%, decreased the shift in UCST to just 21° C. Such magnitudes of this shift could be replicated at neutral pH to maximize CO2-induced pH change. Previous copolymers using DEAEMA have incorporated the monomer at a low mol % of 2-3%. See, Niskanen, J. et al, How To Manipulate The Upper Critical Solution Temperature (UCST), Polym. Chem. 8, 220-232 (2017). The magnitude of the UCST shift increases with increasing mol % of pH- responsive monomer. Based on pH sensitivity and UCST criteria listed above a suitable composition of the triblock ABA copolymer for biosensor 15 is as follows: p(AAm-co-AN-DEAEMA)-b-PDMA525-b-p(AAm-co-AN-DEAEMA), where 525 refers to the length of polymer, with an A block mol% ratio of AAm:AN:DEAEMA is 81.9:15:3.1. The synthesis of copolymer occurs with two rounds of reversible addition-fragmentation chain transfer (RAFT) polymerization, first to synthesize the A-block and then repeated, to attach the A-block to the B-block. Characterizations are performed via gel permeation chromatography (GPC), proton nuclear magnetic resonance (H-NMR), and FT-infrared spectroscopy (FT-IR).

The LCST represents the temperature at which below a polymer is soluble at any mixing ratio of polymer to solvent and above which the polymer is not soluble at high concentration. LCSTs occur in polymers due to attractive ionic or hydrogen bonding interactions between polymer chains due to entropic favorability. At high temperatures, these attractive interactions prevent dissolution of the polymer by water. As temperature decreases, the entropic favorability of mixing allows ionic and hydrogen bonds between chains to break, causing dissolution. Within a hydrogel matrix, whose structure is composed of a polymer network with trapped free-floating aqueous water, dissolution of CO2 into the water, results in the production of hydrogens, as mentioned previously, causing polymer network breakdown and thus a gel to sol phase transition. In the case of p(MEO2MA-co-DMAEMA), hydrogen bonding among DMAEMA monomers (the CO2 sensitive component) is responsible for inducing an LCST shift in the copolymerized MEO2MA monomers. For suitable operation the hydrogel 15 must contain an initial LCST of near 25° C., which causes the copolymer to remain insoluble before overdose. After CO₂ response, the hydrogels’s LCST will decrease below its phase transitions temperature rapidly dissolving the copolymer among the aqueous water in the gel and thereby solubilizing the entire hydrogel. The CO₂ sensitivity of the LCST is a result of the weak polycation, 2-(dimethylamino) ethyl methacrylate (DMAEMA). As CO₂ increases, pH decreases, causing the protonation state of DEAEMA to change, with the tertiary amine picking up a proton and becoming charged. This change in protonation state changes the enthalpy of solvation of the polymer, which causes a shift in the LCST. The shift is especially pronounced near the pKa of the polycation. The amino-methacrylate family of monomers was chosen as the weak polycation monomer because of its near neutral pKa, relative biocompatibility, and numerous previous literature examples demonstrating its potential as a CO₂ specific biosensor. Hydrogen bonding LCST polymers are particularly sensitive to pH due to their low, endothermic enthalpy of solvation. The small magnitude of this enthalpy means that changes to the protonation state of the weak polyelectrolyte have a large impact on the enthalpy of solvation and therefore the LCST. Magnitudes of this shift can be replicated at neutral pH to maximize CO₂-induced changes, and produce a viable hydrogel biosensor. Previous copolymers using aminomethacrylates have demonstrated these large shifts using mol% monomer concentrations as low as 2-3%. The magnitude of the LCST shift increases with increasing mol % of pH or CO₂-responsive monomer. Therefore, the present hydrogel 15 combines the extreme sensitivity of previously demonstrated LCST copolymers with the neutral CO₂ responsiveness of DMAEMA by synthesizing a hypersensitive p(MEO2MA-co-DMAEMA-b-PEO-b- MEO2MA-co-DMAEMA) ABA block copolymer that is 10-20% DEAEMA.

Many other polymer and/or hydrogel materials may be suitable for use as biosensor 15. One potential alternative is a different gel-sol system using bile salt gels (sodium deoxycholate, sodium oleate, sodium erucate, sodium lithocolate, sodium chenodeoxycholate, sodium cholate) which likewise experience a phase transition when exposed to CO₂. However the mechanism of CO₂ activation is different and does not rely on bicarbonate pH changes. For example, sodium deoxycholate (NaDC), a bile salt, is diluted into aqueous water, forming micelles due to its hydrophobic nature, limiting in complete solubility. Upon addition of CO₂ protonation of carboxylate and hydroxyl groups cause hydrogen bonding among neighboring micelles forming bundles of micellular fibers, inducing gel that becomes stronger with the addition of increased CO₂. See, Zhang, M. et al., CO₂ Sequestration By Bile Salt Aqueous Solutions And Formation Of Supramolecular Hydrogels, ACS Sustainable Chemistry & Engineering 7, 3949-3955 (2019). For a nasal clip with naloxone embedded in the gel as per FIG. 2, a CO₂/pH responsive biosensor 15 gel was created using the bile salt sodium oleate with the addition of L-arginine with a specific gaseous CO₂ response range of 6-7% CO₂ for overdose detection and drug delivery, according to the following composition :
Sodium oleate: 100-350 mM (Range)
L-Arginine: 50-200 mM (Range)
Ammonium Chloride 30-100 mM (Range) or added at discretion to change pH of gel solution to reach pH = 9.
Distilled Water

An optimal ratio of L-Arginine to sodium oleate, for fastest response was 1:3, but may change depending on delivery and time requirements. For example, because of the varying duration at which opioids can take effect and thus become an overdose, the hydrogel 15 must be responsive for up to a precautionary 5 hours. After combining all ingredients into solution with magnetic stirring, allow to sit at room temperature for up to 2 hours for gelation to occur. Measured gel transition rates were 0.03 g/2.5 minutes (90 mM arginine) to 0.02 g/7.5 minutes (120 mM arginine). Under 13% CO₂ concentrations in CO₂/N₂ gas blend at a rate of 1.5 L/min. The ingredient of the gel sensor 15, for varied CO₂ responses between 1%-10%, under conditions of temperature and humidity of 30-100% relative humidity, and 20-40° C. respectively, as well as cyclic and diminishing rates between 4-20 breaths per minute (ventilation rate 0.5 L/min-10 L/min).

One skilled in the art should understand that the above-described biosensor 15 is but one example, and many other modes of polymer research have been used for CO₂ detection and response.

The biosensor 15 encapsulates, embeds or otherwise occludes a 0.1-1 mL solution dose of medication for intranasal administration preferably having a naloxone-concentration within a range of from 0.4-20 mg/0.1 mL, either aerosolized, liquid or powdered, Recent research on intranasal naloxone delivery has demonstrated that physical application of naloxone to the inferior turbinates in the nature of a swab or applicator, may absorb with equal to or greater efficiency than intranasal spray. In an embodiment the active ingredient for overdose reversal may be a powdered solution of naloxone hydrochloride dihydrate diluted in aqueous water, with one or more excipients as follows:
Benzalkonium chloride (preservative).
Na2-EDTA (stabilizer).
NaCl (pH adjuster).

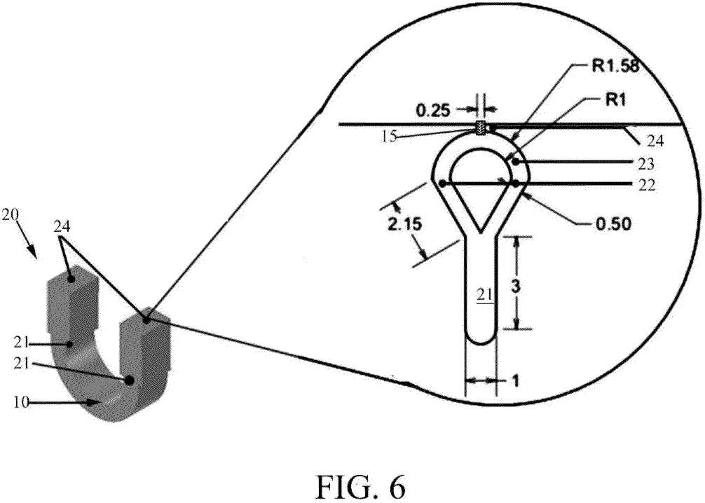

FIG. 6 illustrates an alternate embodiment of the device 20, still a wearable nasal clip generally U-shaped with a unitary two-pronged housing 10 with spaced prongs 11 that grip the septum, but rather than opposing drug reservoir chambers 12 containing hydrogel-embedded naloxone, the naloxone 16 in each prong is contained in a chamber 21 that leads to two separate branches 22 that converge at conjoining passage 23 which leads to a nozzle 24 that is occluded by the biosensor 15. The dual branches 22 provide for failsafe delivery, preferably in aerosol form. Where aerosol delivery is desired the chamber 21 is pressurized with about 30 psi (~200 kPa) of pressure, which is known to be enough to deliver a standard full does of naloxone (naloxone-HCl 4 mg/0.1 mL) up to 2-5 cm in vertical height, for delivery into the inferior and middle nasal turbinates of the nasal passageway (conventional administration of naloxone is aerosol by a manually actuated push-plunger generally producing 30 psi). Due to the varying duration at which opioids can take effect, the drug chamber 21 must also retain pressure to ensure successful delivery at all possible times of onset of overdose, from 15 minutes to 4 hours after use. Therefore the entire device 20 must retain appropriate pressure for up to a precautionary 5 hours. The biosensor 15 may be liquid-injected into the nozzle/outlet 24 and left to cool into a gel. Below the adjoining bridge 23 chamber 21 may be pre-pressurized, or else by a pressurizing mechanism such as a one-way micro-plunger. Exemplary flow path dimensions are as follows: length of branches 22 are 2.15 mm (length) × 500 um (wide) connected to a radial flow path 23 near the outlet with a radius of 1.58 mm. The nozzle outlet 24 is 250-500 um wide. The height/thickness of the branched channels 22 are 500 um.

For a nasal clip with pressurized chambers the gel may be impregnated with CO₂ (40 mM) to obtain the maximum modulus of gel, forming the biosensor plug 15 at the outlet 24. Regular exhalation produces negligible CO₂ to affect the molds of the gel, yet with the occurrence of an overdose, the increase in expelled CO₂ weakens the gel, eventually allowing it to be jettisoned from the loaded pressure in the drug reservoir 11.

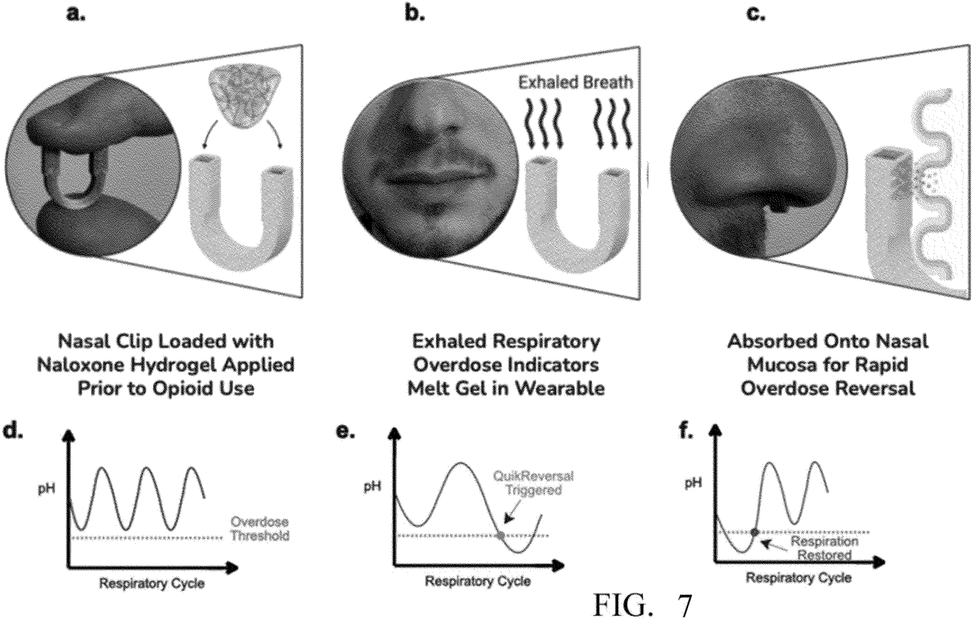

FIG. 7 illustrates a sequential method of using devices 2, 20 and mechanism of CO₂ activation.

As seen at (a), the device 2, 20 is loaded with naloxone-embedded hydrogel prior to use. If the device is pressurized as above, each drug reservoir 12, 21 may be pressurized up to 30 psi by a loading mechanism that is detached from the device 2, 20 prior to application.

At (b) if an overdose occurs, the biosensor 15 is ultrasensitive to pH changes, and produces a reverse gel-sol phase transition from a gel state to a liquid state. At (c) the biosensor 15 unblocks the release of naloxone into the nasal passages. The respiratory cycle corresponding to steps (a-c) are shown below at (d-f), respectively. The outlet nozzles 13, 24 preferably release droplets of 30-100 um in a fanned profile.

It should now be apparent the above-described intranasal device for automatic opioid overdose detection and naloxone delivery, triggers automatically in response to physical symptoms of an overdose without the need for bystander intervention. This provides a safety-net for those at risk of opioid overdose. The device will offer product users the autonomy to prevent their own accidental overdose, without relying on bystanders or emergency personnel response.

Unlike other drug delivery mechanisms, such as mechanical or electrical, chemical methods of delivery provide a biocompatible alternative, with discrete, cheap, and noninvasive benefits. To date, there are no medication delivery device or method is capable of actively and automatically intervening during an overdose event. The present disclosure does, and holds promise to curb fatal opioid overdose and give individuals affected from OUD the ability to achieve lasting sustainable treatment and recovery, ultimately preventing over 60,000 deaths annually.

The foregoing disclosure of embodiments of the present invention has been presented for purposes of illustration and description. It is not intended to be exhaustive or to limit the invention to the precise forms disclosed. Many variations and modifications of the embodiments described herein will be obvious to one of ordinary skill in the art in light of the above disclosure. The scope of the invention is to be defined only by the claims, and by their equivalents.

What is claimed is:

1. A device for automatic symptom detection and medication delivery, comprising:
a housing configured to be worn by a patient, said housing defining an internal medication chamber configured for containing a dose of medication, and at least one port in ambient fluid communication with said medication chamber; and
a biosensor constraining a dose of naloxone within the internal medication chamber, the biosensor comprising a phase-transition material responsive to a symptom of said patient and configured to transition from a solid state to a non-solid state in response to said symptom, thereby releasing said medication through said at least one port.

2. The device according to claim 1, wherein said housing is configured to be worn intranasally.

3. The device according to claim 2, wherein said housing comprises a unitary member having two spaced prongs configured to grip the septum for wearing thereon.

4. The device according to claim 1, wherein said housing comprises two internal drug reservoirs.

5. The device according to claim 1, wherein said housing comprises a plurality of ports all in ambient fluid communication with said medication chamber.

6. The device according to claim 1, further comprising a dose of naloxone occupying said medication chamber.

7. The device according to claim 1, wherein said biosensor comprises a phase-transition material responsive to a symptom of said patient and configured to transition from a solid state to a non-solid state in response to a change in nasal CO₂ levels in exhaled breath.

8. The device according to claim 1, wherein said biosensor comprises a hydrogel.

9. The device according to claim 8, wherein said hydrogel biosensor encapsulates the dose of medication.

10. The device according to claim 8, wherein said biosensor comprises a block copolymer.

11. The device according to claim 10, wherein said biosensor comprises an ABA block copolymer.

12. The device according to claim 8, wherein said biosensor comprises a bile salt.

13. The device according to claim 8, wherein said biosensor comprises sodium oleate (NaOA).

14. The device according to claim 1, wherein said at least one port in ambient fluid communication with said medication chamber comprises an aerosolizing nozzle.

15. An intranasal device for automatic opioid overdose detection and naloxone delivery, comprising:
a unitary housing formed with two spaced prongs configured to grip the septum for wearing thereon, the housing including an internal medication chamber configured for containing a dose of medication, and at least one port in ambient fluid communication with said medication chamber; and
a biosensor constraining a dose of naloxone within the internal medication chamber, the biosensor comprising a phase-transition material sensitive to CO₂ and configured to transition from a solid state to a non-solid state in response to a change in exhalation levels of CO₂, thereby unblocking said at least one port.

16. The device according to claim 15, wherein said housing comprises two internal drug reservoirs.

17. The device according to claim 15, wherein said housing comprises a plurality of ports all in ambient fluid communication with said medication chamber.

18. The device according to claim 15, wherein said biosensor comprises a block copolymer.

19. The device according to claim 8, wherein said biosensor comprises a bile salt.

20. An intranasal device for automatic opioid overdose detection and naloxone delivery, comprising:
a unitary housing formed with two spaced prongs configured to grip the septum for wearing thereon, and two distal medication chambers each configured at the end of a respective prong, configured for containing a dose of medication, and each medication container having a plurality of ports in ambient fluid communication; and
a hydrogel biosensor occluding said plurality of ports, the biosensor comprising a phase-transition material sensitive to CO₂ and configured to transition from a solid state to a non-solid state in response to a change in exhalation levels of CO₂, thereby unblocking said medication chambers.
